(19) United States
(12) Patent Application Publication
(10) Pub. No.: US 2014/0162248 A1
(43) Pub. Date: Jun. 12, 2014

(54) Use of Aptamers in Liquid Chromatography and Liquid Chromatography - Mass Spectrometry

(71) Applicants: Steven A. Cohen, Hopkinton, MA (US); Martin Gilar, Franklin, MA (US)

(72) Inventors: Steven A. Cohen, Hopkinton, MA (US); Martin Gilar, Franklin, MA (US)

(73) Assignee: Waters Technologies Corporation, Milford, MA (US)

(21) Appl. No.: 14/101,624

(22) Filed: Dec. 10, 2013

Related U.S. Application Data
(60) Provisional application No. 61/735,120, filed on Dec. 10, 2012.

(51) Int. Cl. C12N 15/115; C12Q 1/68

(52) U.S. Cl. CPC C12N 15/115; C12Q 1/6876
USPC 435/6.1; 536/23.1; 530/417

(57) ABSTRACT

The present disclosure relates to the use of aptamers in solid phase extraction, chromatography and chromatography-mass spectrometry systems. More specifically, the present disclosure relates to the use of aptamers in SPE and chromatography systems to selectively retain, extract and/or pre-concentrate target molecule(s) having a specific affinity for the particular aptamer(s). The target molecule(s) can be further analyzed by mass spectrometry with limited interferences and/or enhanced sensitivity.

### CROSS-REFERENCE TO RELATED APPLICATIONS

This application claims priority to U.S. Provisional Application No. 61/735,120, filed Dec. 10, 2012, which is incorporated herein by reference in its entirety.

### FIELD OF THE TECHNOLOGY

The present disclosure relates to the use of aptamers in solid phase extraction (SPE), chromatography and chromatography-mass spectrometry systems. More specifically, the present disclosure relates to the use of aptamers in SPE and chromatography systems to selectively retain, extract and/or pre-concentrate target molecule(s) (e.g., a group of proteins) having a specific affinity for the particular aptamer(s). The target molecule(s) can be further analyzed by mass spectrometry, wherein such analysis has limited interferences and/or enhanced sensitivity.

### BACKGROUND

Aptamers are typically 30 to 60 nucleotides-long oligonucleotide sequences that have an affinity towards proteins, or other molecules of interest. Aptamers are generally not as selective as monoclonal antibodies, but they are generally more selective than sorbents used for solid phase enrichment. Aptamer affinity can also be enhanced by chemical modification of the putative aptamer sequence in order to achieve a high binding affinity comparable monoclonal antibodies (mAb, which can have dissociation constants Kd in ˜nM range). To date, aptamers have not been used with SPE or chromatography systems to selectively retain, extract and/or pre-concentrate target molecule(s) having a specific affinity for the particular aptamer(s).

### SUMMARY

The present disclosure relates to the use of aptamers in solid phase extraction, chromatography and chromatography-mass spectrometry systems.

In one embodiment, the present disclosure relates to a solid phase extraction system for isolating at least one analyte from a liquid sample comprising a solid support having a plurality of aptamers having an affinity for the at least one analyte.

In another embodiment, the present disclosure relates to a chromatographic system for separating at least one analyte from a liquid sample comprising a chromatography column having a plurality of aptamers having an affinity for the at least one analyte.

In another embodiment, the present disclosure relates to a method for analyzing a liquid sample for at least one analyte, the method comprising contacting the liquid sample and a plurality of aptamers immobilized on a solid support, the aptamers having an affinity for the at least one analyte, thereby selectively adsorbing the at least one analyte, if present, to the solid support; removing impurities from the liquid sample not adsorbed to the solid support; and selectively desorbing the at least one analyte from the solid support.

In another embodiment, the present disclosure relates to a method of separating at least one analyte from a liquid sample, the method comprising placing the sample in a chromatography system having a chromatography column with a plurality of aptamers having an affinity for the at least one analyte, thereby selectively adsorbing the at least one analyte, if present, to the chromatography column; and eluting the sample from the chromatography column.

These embodiments can further include mass spectrometry instrumentation and analysis by such mass spectrometry instruments.

### BRIEF DESCRIPTION OF THE DRAWING

FIG. 1 shows an exemplary flow diagram of a solid phase extraction system comprising aptamers.

FIG. 2 shows an exemplary flow diagram of a chromatography—spectrometry system comprising aptamers.

### DETAILED DESCRIPTION

The present disclosure relates to the use of aptamers in solid phase extraction, chromatography and chromatography-mass spectrometry systems. In one embodiment, the present disclosure relates to a solid phase extraction or chromatography system for isolating or separating at least one analyte from a liquid sample comprising a solid support or chromatographic column having a plurality of aptamers having an affinity for the at least one analyte.

The solid phase extraction or chromatography system includes high pressure liquid chromatography, supercritical fluid chromatography, carbon dioxide based chromatography, subcritical fluid chromatography, gas chromatography and related systems and techniques. The solid support and chromatographic column can be any known support or column capable of being used with aptamers for isolating or separating at least one analyte from a liquid sample.

The plurality of aptamers can be characterized by a K_{d} having a value of about 10⁻⁹, 10⁻⁸, 10⁻⁷, 10⁻⁶, 10⁻⁵, 10⁻⁴, 10⁻³, 10⁻², or 10⁻¹. The aptamers can have individual lengths of about 60, 55, 50, 45, 40, 35, 30, 25, 20, 15, 10, or 5 monomers. The plurality of aptamers can be substantially homogeneous or include different aptamer sequences (e.g., multiple sequences directed to the same and/or different analytes). In one example, the plurality of aptamers can also employ a tandem hybridization approach (e.g., two or more short oligonucleotides working in tandem towards a specific complementary molecule).

The plurality of aptamers can be highly specific for a particular analyte. Alternatively, the plurality of aptamers can be cross reactive with at least one derivative, metabolite, or analog of the analyte (e.g., where the at least one analyte comprises a molecule of interest in addition to at least one derivative, metabolite, or analog thereof).

Aptamers according to the present disclosure can be based on native polymer sequences, which can be subsequently chemically modified (or not modified) before use. For example, aptamers can be nucleic acids with negatively charged phosphate backbones. Such aptamers can exhibit cation exchange properties, which can be undesirable in some applications (e.g., they will interact with any positively charged proteins). In various embodiments, aptamers according to the present technology can have neutral or positive backbones (e.g., methylphosphonate backbone or PNA-peptide nucleic acid). Neutral backbones can facilitate immobilized aptamer selectivity at low ionic strength.

One advantage of using aptamers in these systems is their straightforward synthesis (i.e., given a predetermined sequence). In general, unmodified aptamers have lower affinity towards target molecules compared to monoclonal antibodies (mAbs). Typical aptamer K_{d} values are in the ˜μM range, which can result in a relatively high desorption speed (e.g., three orders lower affinity compared to mAbs). High affinity is desirable in ELISA or other assays using affinity selection. The enrichment of analytes is typically achieved in a batch type of experiment. The washes used to remove unbound or weakly bound molecules from the immobilized target can result in minor loss of analyte. The loss is more significant if the selector with higher dissociation constant K_{d} is used. Displacement of the target analyte by competing molecules can also occur with lower affinity aptamers.

With respect to affinity, the lower the dissociation constant K_{d} is, the stronger the interaction is with the target. In the nM range the desorption speed is 10⁹ fold slower than adsorption speed. For K_{d}=1 the desorption and adsorption speeds are identical. In the case of a separation with a media having such an affinity, each wash cycle will remove a substantial amount of bound molecule. In various embodiments, one goal of aptamer selection can be to prepare sequences with highest possible affinity towards a target molecule while maintaining the lowest possible affinity towards the remaining components in a sample.

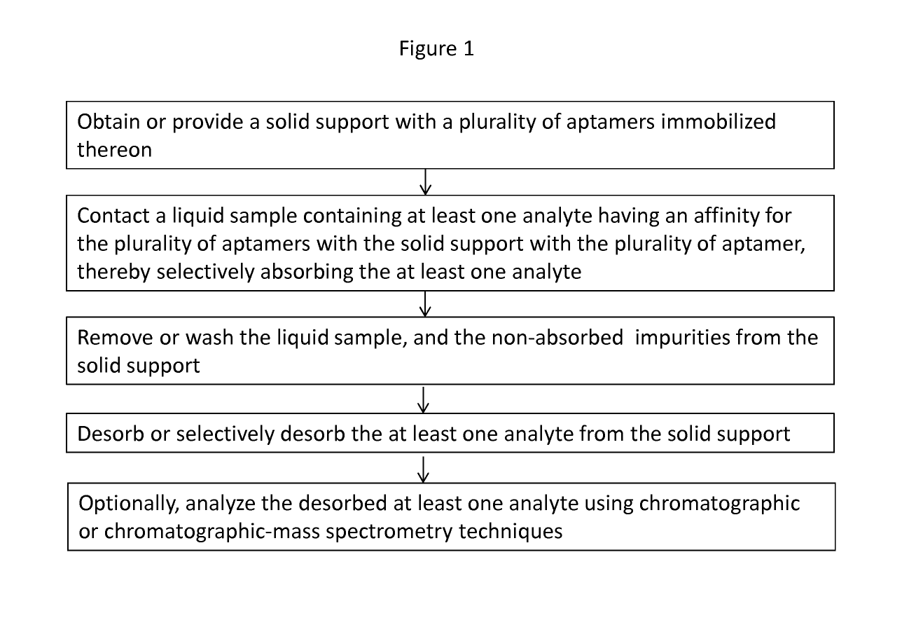

In another embodiment, the present disclosure relates to a method for analyzing a liquid sample for at least one analyte, the method comprising contacting the liquid sample and a plurality of aptamers immobilized on a solid support, the aptamers having an affinity for the at least one analyte, thereby selectively adsorbing the at least one analyte, if present, to the solid support; removing impurities from the liquid sample not adsorbed to the solid support; and selectively desorbing the at least one analyte from the solid support. FIG. 1 shows an exemplary flow diagram of the method.

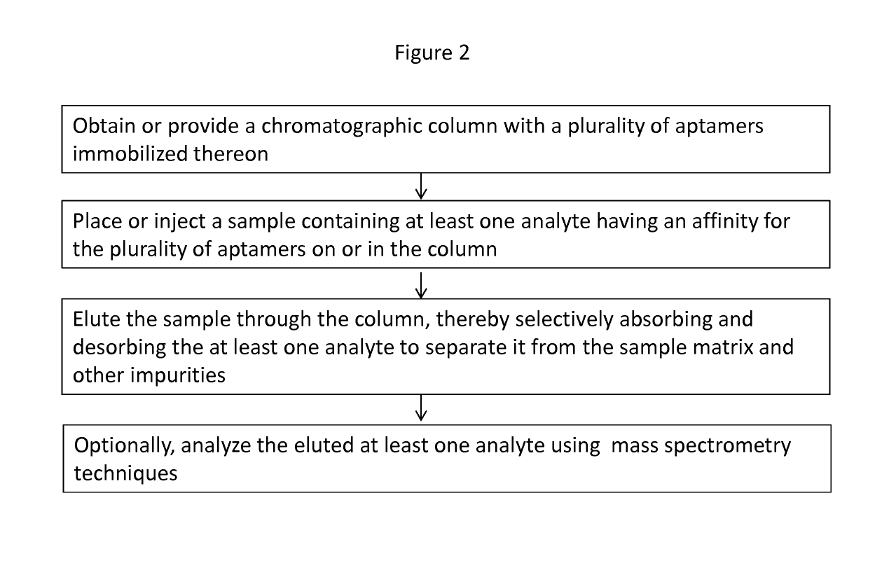

In a related embodiment, the present disclosure relates to a method of separating at least one analyte from a liquid sample, the method comprising placing the sample in a chromatography system having a chromatography column with a plurality of aptamers having an affinity for the at least one analyte, thereby selectively adsorbing the at least one analyte, if present, to the chromatography column; and eluting the sample from the chromatography column. FIG. 2 shows an exemplary flow diagram of the method.

Conventional chromatography uses generic selectors (e.g., C18 chemistry). Generic solid phase enrichment sorbents us similar material, both of which have K_{d} values in order of 10⁻¹-10⁻³ (e.g., for small molecules). In one embodiment, this is sufficient affinity for retention and enrichment of target analyte(s) in these applications because chromatography offers a chance for repeated adsorption and desorption events. While the interaction is not selective and many molecules are retained, the chance for selective enrichment increases with the number of theoretical plates. Because of limited selectivity of SPE, the target analyte can be contaminated and lengthy SPE method development is required. However, the subsequent chromatographic (or LC-MS) analysis further separates the analyte signal from background of contaminants, which is a reason why the SPE and LC techniques are widely and successfully used for sample preparation and analysis. If the sample matrix becomes very complicated and target analytes are present in minor concentrations, generic SPE reaches its limits. Therefore, the present disclosure also relates to affinity sorbents (e.g., aptamers) with higher affinity characteristics. Aptamers are also distinct from other affinity sorbents, such as mAbs and imprinted polymers, which can be difficult and expensive to prepare (e.g., because they offer selectivity only towards the target molecule, a unique sorbent has to be prepared for each target molecule, which can be expensive and cumbersome). Aptamers can be prepared with a selectivity towards a groups of related molecules and are less expensive to prepare.

In various embodiments of the present disclosure, aptamers can be adapted for use as selectors for sample preparation in SPE or for general use in liquid chromatography. Aptamers can also be adapted for selective enrichment of target molecules from complex samples despite having a lower binding affinity than mAbs. Aptamers having a K_{d} in the ˜mM-nM range provides a useful selectivity towards the target, especially when combined with SPE and LC techniques. Because of lower useful K_{d} range requirement, it is easier to identify new aptamer sequences (e.g., specific to a target) in accordance with the technology.

Shorter aptamers can be adapted for use as selectors for SPE and LC. There is a relationship between length and affinity of aptamers, such that longer sequences are more selective and more expensive. SPE and LC in accordance with the present disclosure can use relatively short and inexpensive aptamers. The aptamers of the present disclosure can have individual lengths of about 60, 55, 50, 45, 40, 35, 30, 25, 20, 15, 10, or 5 monomers.

Without wishing to be bound to any particular theory, it is believed that the combination of aptamers (moderate, but having sufficient affinity towards the target molecule) and repetitive adsorption-desorption events provided by SPE and LC techniques can facilitate advantageous separation methods and compositions of the technology. For example, aptamer-SPE sample preparation devices in accordance with the present disclosure can selectively enrich the target analytes while suppressing the sample background to sufficient level. Many aptamers can be quickly prepared and immobilized on the same or on separate sorbents for SPE experiments. A generic SPE sorbent (e.g., activated with binding chemistry) can be used and a specific sorbent can be prepared on-demand by first adsorbing (binding) the selector-aptamer of desirable nature. Single SPE sorbents can be modified for different types of applications by binding a selected aptamer from predefined aptamer library.

Nucleic acid aptamers can also be used. These aptamers are capable of selective, tight binding for a variety of molecules. The generation of a highly selective, very tight binding aptamer absorbant can be a difficult process. Synthesizing the resulting tight binding aptamers can be expensive due to the length of the chain required to produce the proper three dimensional structure. In some embodiments, the highly selective tight binding aptamers can be used in aptamer applications such as ELONA (i.e., enzyme-linked oligonucleotide assay), an ELISA equivalent assay.

Using aptamers to capture and enrich reagents prior to analysis of a bound fraction by LC-MS is easier and requires aptamers having a relatively lower affinity than the affinity requirements for an ELONA and similar biosensor applications. The separation power of both LC and MS makes it feasible to analyze much more complex mixtures. The required selectivity of binding required for an ELISA type assay is not necessary for LC/MS analysis. Aptamers with relaxed selectivity requirements, or lower affinity values to a target molecule(s), can be shorter, require less modification and/or are less expensive to synthesize than tight binding aptamers. The present disclosure relates to both lower affinity aptamers and higher affinity aptamers for general and specific SPE, chromatographic, and chromatographic-mass spectrometry systems, including affinity based systems.

In another embodiment, aptamers can be used for sample enrichment of trace components in complex matrices such as biofluids, food and environmental samples with SPE, LC or LC-MS analysis. The aptamers can be those with less than ideal selectivity and lower binding affinities than, for example, monoclonal antibodies. A variety of solid supports may be used to analyze different complex matrices. For example, a solid support or capture system for these matrices can include binding the aptamers to magnetic beads, SPE device, etc.

The present disclosure can also provide aptamers having an affinity/specificity adapted for a specific use. For example, higher affinity/specificity aptamers can be adapted for separations (e.g., low abundance peptides and/or proteins) whereas lower affinity/specificity aptamers can be adapted for cleanup (e.g., high abundance interferences). In various embodiments, negative selection can be used to simplify a sample matrix from high abundance interferences. Different aptamers can be used that bind a desired target as well as many similar interfering molecular structures. The selective use of these aptamers can be used to remove highly abundant interferences from a system. For example, the sample matrix can be passed through a capture system having aptamers with an affinity to the interfering molecules and little or no affinity for the target molecules. The resulting simplified mixture can then be subjected to LC-MS analysis.

The at least one analyte may be any analyte, or target molecule(s), having an affinity for the aptamer(s). In embodiments relating to plasma proteomics, the targeted analytes can be proteins or signature peptides. In other embodiments, clinical markers such as cytokines, vitamin D and steroids can benefit from aptamer capture in accordance with the present disclosure prior to LC/MS analysis. In some embodiments, protein bioanalysis (analysis of protein therapeutics in biofluids) can also benefit from a modestly selective enrichment whereby the desired structure and related components (modified forms, degraded forms etc.) are also captured. In certain embodiments, other complex matrices (foods, environmental samples) are also amenable to aptamer capture of trace analytes such as pesticides, endocrine disruptors, and pharmaceutical compounds in wastewater with subsequent LC/MS analysis.

Aptamers in accordance with the present disclosure can be adapted for various qualitative, quantitative, and semiquantitative methods (e.g., TOF and TQ MS). Different combinations of aptamer-based SPE devices with differing ranges of selectivities can be useful depending on the method.

The disclosures of all cited references including publications, patents, and patent applications are expressly incorporated herein by reference in their entirety.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

While this disclosure has been particularly shown and described with reference to example embodiments thereof, it will be understood by those skilled in the art that various changes in form and details may be made therein without departing from the scope of the invention encompassed by the appended claims.

We claim:

1. A solid phase extraction system for isolating at least one analyte from a liquid sample comprising a solid support having a plurality of aptamers having an affinity for the at least one analyte.

2. A chromatographic system for separating at least one analyte from a liquid sample comprising a chromatography column having a plurality of aptamers having an affinity for the at least one analyte.

3. A method for analyzing a liquid sample for at least one analyte, the method comprising
(i) contacting the liquid sample and a plurality of aptamers immobilized on a solid support, the aptamers having an affinity for the at least one analyte, thereby selectively adsorbing the at least one analyte, if present, to the solid support;
(ii) removing impurities from the liquid sample not adsorbed to the solid support; and
(iii) selectively desorbing the at least one analyte from the solid support.

4. A method of separating at least one analyte from a liquid sample, the method comprising
(i) placing the sample in a chromatography system having a chromatography column with a plurality of aptamers having an affinity for the at least one analyte, thereby selectively adsorbing the at least one analyte, if present, to the chromatography column; and
(ii) eluting the sample from the chromatography column.

5. The method of claim 3, further comprising the step of analyzing the at least one analyte using mass spectrometry.

6. The method of claim 4, further comprising the step of analyzing the at least one analyte using mass spectrometry.
